(19) United States
(12) Patent Application Publication
(10) Pub. No.: US 2005/0037064 A1
(43) Pub. Date: Feb. 17, 2005

(54) Heteroxylan film-forming composition for making capsules and resulting capsules

(75) Inventors: Serge Basquin, Toulouse (FR); Fabien Darasse, Albi (FR); Denis Despre, Riom (FR); Arnaud Messager, Riom (FR)

Correspondence Address: JAMES C. LYDON, 100 DAINGERFIELD ROAD, SUITE 100, ALEXANDRIA, VA, 22314, US

(73) Assignee: Ulice and Pierre Fabre Medicament

(21) Appl. No.: 10/500,208

(22) PCT Filed: Dec. 26, 2002
(86) PCT No.: PCT/FR02/04565

(30) Foreign Application Priority Data
Dec. 27, 2001 (FR) 01/16951
Oct. 23, 2002 (FR) 02/13235

(51) Int. Cl. A61K 31/715; A61K 9/48

(52) U.S. Cl. 424/451; 514/54

(57) ABSTRACT

A heteroxylan composition for preparing capsules or tablets, in particular soft capsules or hard tablets, the composition containing at least a heteroxylan compound, at least a softening agent, and at least a gelling agent. Also disclosed is a film obtained from the composition, and capsules obtained from the composition or the film.

The field of the present invention relates in general to film-forming compositions. More particularly, the present invention relates to a composition based on heteroxylans for the preparation of capsules.

The expression capsules is understood to mean hard or soft capsules, which are devices which are widely used nowadays to contain pharmaceutical, phytotherapeutic or food products.

Pharmaceutical hard capsules, which are generally oblong, are classified as solid dosage forms (BOWMAN & OFNER, 2002) intended mainly for the ingestion of unit doses of solid active principles by contrast to soft capsules used for liquid or semisolid medicaments.

Capsules also make it possible to preserve products whose taste and/or odor can prove unpleasant.

The preparation of hard capsules is traditionally carried out based on animal gelatin, to which there may be added additives such as plasticizers, colorings, preservatives and the like.

The preparation of these gelatin-based devices is described in “Pharmacotechnie Industrielle” (Rosetto, 1998, Ed. IMT).

However, since the appearance of public health problems linked to Bovine Spongiform Encephalitis (BSE) and the discovery of its vector in animal tissues which are traditionally used to isolate gelatin, the scientific community and industrialists in the technical field have become conscious of the risk arising from the use of gelatin of animal origin in products intended to be ingested.

The development of a product capable of replacing gelatin has therefore become an important area of research for many companies in the technical field.

Gelatin substitutes have therefore been envisaged, such as starches: using an extrusion process, it is possible to produce starch capsules industrially (Targit® Technologies, VILIVALAM et al., 2000).

A patent has also been filed proposing the manufacture of hard capsules from κ-carrageenan as main film-forming agent, combined with a variety of other hydrocolloids such as gellan gum and mannans (U.S. Pat. No. 6,214,376), but this formula does not yet have an industrial future.

Research studies have also been carried out in order to develop films whose formulation is based on cellulose ethers and which have the same mechanical properties and barriers to gases and to lipids as gelatin films (KAMPER & FENNEMA, 1985).

Indeed, native cellulose, which is predominantly extracted from plant cell walls, is insoluble in water because of an excessively large quantity of intramolecular hydrogen bonds within the polymer and a high degree of crystallinity which limits its solvation.

On the other hand, by introducing along the chain substituents which interfere with the formation of the crystalline units, it then becomes possible to solvate this polymer: this is achieved by etherification.

Thus, by reacting cellulose with a sodium hydroxide solution and then with methyl chloride, propylene oxide or sodium monochloroacetate, methylcellulose (MC), hydroxypropyl methylcellulose (HPMC), hydroxypropyl cellulose (HPC) and carboxymethylcellulose sodium (CMC) are produced.

These compounds consequently make it possible to produce films which are transparent, flexible but solid, soluble in water and resistant to oils and fats (NISPEROS-CARRIEDO, 1994).

Of these cellulose derivatives, HPMC most particularly is used as gelatin substitute in pharmaceutical hard capsule applications.

Used industrially as sole gelling agent (EP-A-0 056 825) or combined with carrageenans (EP-A-0 592 130, EP-A-1 029 539), it makes it possible to obtain capsules with the same properties as gelatin capsules except for the rates of dissolution, which are lower.

However, while these capsules are technically and commercially advantageous, they have nevertheless a defect: as chemical derivatives, interactions can be expected with certain active compounds which the capsules may contain. The principle of caution would therefore require that their use in the food industry be avoided.

The pharmaceutical and food industries are still waiting for capsules in which the gelatin has been replaced by one or more constituents of plant origin, which do not cause any risk for the consumer, whose manufacture does not result in any unacceptable additional cost compared with gelatin-based capsules, and which possess mechanical and dissolution properties of the same type as the latter.

It is therefore to the applicant's credit to have demonstrated that the use of heteroxylans, in particular of arabinoxylans, as film-forming constituents in a composition for manufacturing hard or soft capsules, could constitute a novel opening and an advantageous alternative to the use of the abovementioned compounds, in particular in terms of safety, cost of manufacture, and quality of the resulting films.

It happens to be the case that heteroxylans are present in a large quantity in corn brans (peripheral part of corn seeds), a by-product of the corn milling industry, but they are also found in a significant quantity in rye and rice brans. The majority of these corn brans are currently intended as animal feed, and a very small quantity is used as a source of dietary fiber. Corn brans consist mainly of cellulose (10 to 20%) and of heteroxylans (40 to 50%). High yields of extraction (up to 90%) of the heteroxylans contained in corn bran can be obtained with no apparent reduction in the molecular mass of the polymer.

Heteroxylans are plant polysaccharides which are located in the cell walls (parietal polysaccharides) and belong to the hemicellulose group. They are the most abundant non cellulosic parietal polysaccharides. They comprise a linear backbone of β-1,4-linked xylopyranoses substituted with side chains, varying in type and number. The β-1,4-type glycol bond gives the chain a relatively stretched conformation. The helix conformation of the β-1,4 xylane is more flexible than that of cellulose, despite a similarity between xylose and glucose, because it is stabilized by only a single hydrogen bond whereas there are two of them in the case of cellulose. This bond is established between the hydrogen of the hydroxyl group at the 3-position of a xylose residue, and the oxygen at the 5-position of the next. When xyloses are substituted, they are substituted on their oxygen at the 3-position and more rarely on their oxygen at the 2-position. The nature of the side chains, their proportion and their mode of branching on the xylose backbone are structural elements which differ from one heteroxylan to another.

In the heteroxylans obtained from corn brans, xylose constitutes about half of the monosaccharides present, arabinose about a third, hence their name arabinoxylans. Galactose, glucuronic acid and ferulic acid are the other constituents thereof. The molecular mass of heteroxylans varies between 100 000 and 250 000 g/mol, this variability being explained in particular by the differences in the mode of extraction used or in the method of analysis used in order to determine the sugars constituting the heteroxylan analyzed. Their degree of polymerization is therefore between 700 and 1800.

Heteroxylans are generally extracted in an alkaline medium; depending on the variants of the method of extracting heteroxylans, three main categories of heteroxylans may be obtained, namely grade C, B or A heteroxylans, which correspond to unpurified, moderately purified and highly purified products, respectively.

A first objective of the present invention is therefore to provide a composition intended to form a film used for the manufacture of capsules which can be used in the pharmaceutical, phytotherapeutic or food sector.

A second objective of the invention is to provide a film having the best possible visual appearance, capable of being molded and intended to be used for the manufacture of capsules.

A third objective of the invention is to obtain capsules from the present composition or the present film.

These objectives, among others, are achieved by the present invention which relates to a film-forming composition for the manufacture of capsules, comprising:
at least one compound of the heteroxylan type,
at least one plasticizing agent, and
at least one gelling agent.

Preferably, such a composition is intended for the production of hard capsules.

Remarkably, the heteroxylan used in this composition is arabinoxylan.

Advantageously, the plasticizer is preferably chosen from the group comprising (poly)hydroxylated compounds, and more preferably from the group consisting of glycerol, sorbitol, polyethylene glycol, propylene glycol, maltitol, triacetin or mixtures thereof.

Advantageously, this gelling agent, preferably of plant origin, is selected from the group comprising carrageenans (κ- and ι-carrageenans), gellan gum, pectins or mixtures thereof.

According to a preferred variant, the composition on a dry basis comprises:
between 60 and 99% by weight of arabinoxylan,
between 5 and 40% by weight of plasticizer, and
between 0.1 and 20% by weight of gelling agent.

More advantageously still, the composition additionally comprises at least one bulking agent. This bulking agent is chosen from the group comprising carbohydrates such as sucrose, fructose, starch, cellulose, maltodextrins, cereal and noncereal flours, mineral fillers such as calcium, sodium or potassium salts or mixtures thereof. Such a bulking agent may advantageously be a maltodextrin having a Dextrose Equivalent value of 5 to 40.

More preferably, the bulking agent is contained in the composition in a proportion of between 0 and 70% by dry weight.

Remarkably, the arabinoxylan is preferably extracted from corn, rye or rice brans, or mixtures thereof.

According to another advantageous variant, the composition according to the invention additionally comprises an additive or a mixture of additives chosen from:
colorings, chosen in particular from the group consisting of titanium oxide, iron oxide, patent blue, quinoline yellow, orange yellow S, cochineal red A or chlorophillin-copper complex,
antioxidants such as ascorbic acid, tocopherol, butylhydroxyanisole (BHA) or butylhydroxytoluene (BHT).

Thus, the composition comprises:
between 0 and 3% by dry weight of coloring, and/or
between 0 and 3% by dry weight of antioxidant.

Remarkably, the composition is provided in the form of a solution, preferably an aqueous solution.

According to this remarkable characteristic, the composition comprises from 25 to 80% by weight of water.

Another subject of the invention relates to the use of the abovementioned composition for producing a film.

Another subject of the invention relates to a film obtained from the composition or based on the use of such a composition.

According to a remarkable characteristic, this film has the following mechanical properties:
a rupture strength of between 30 and 250 N,
an elasticity of between 20 and 120 N.s⁻¹,
a deformation of between 2 and 20%.

Yet another subject of the invention relates to a capsule obtained from the composition or from a film according to the invention.

These capsules may be hard or soft capsules.

Advantageously, soft heteroxylan capsules are produced from a composition containing a large quantity of plasticizer and very little or no gelling agent. Such a capsule contains for example:
between 60 and 99% by dry weight of at least one heteroxylan,
between 5 and 40% by dry weight of at least one plasticizer,
between 0.1 and 20% by dry weight of at least one gelling agent,
between 0 and 70% of at least one bulking agent.

The capsules thus obtained have a final moisture of between 5 and 18%.

Hard heteroxylan capsules are, for their part, produced from a composition containing, by contrast, little plasticizer and generally more gelling agent. Thus, a hard capsule may contain:
between 20 and 90% by dry weight of at least one heteroxylan,
between 10 and 30% by dry weight of at least one plasticizer,
between 5 and 20% by dry weight of at least one gelling agent,
between 0 and 70% by dry weight of at least one bulking agent.

The capsules thus obtained generally have a final moisture of between 5 and 18%.

The method used for the preparation of these capsules is chosen from methods known to a person skilled in the art and which are customarily used.

The invention will be understood more clearly with the aid of the following examples, which are not at all limiting, with reference to the drawings in which:

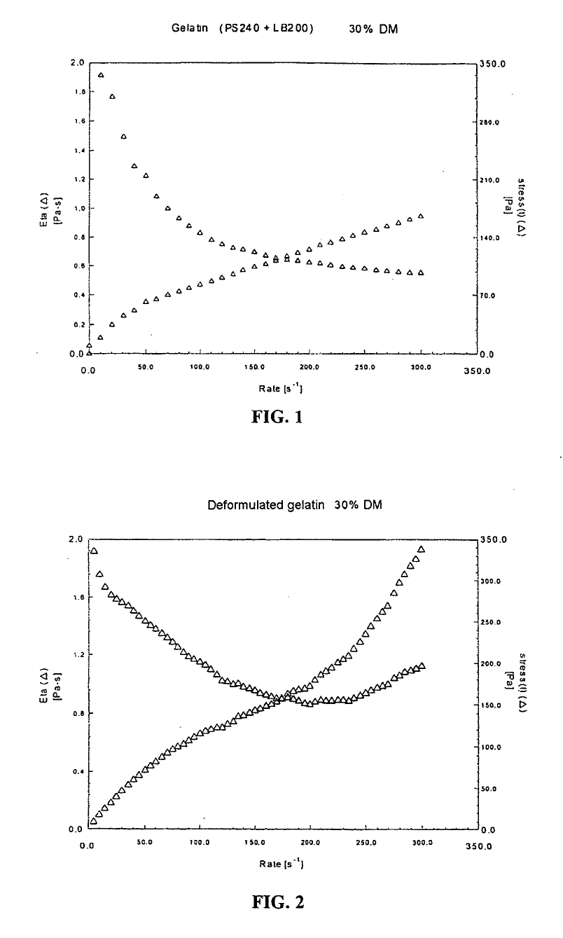

FIG. 1 represents the flow curve for a gelatin-based steep solution containing 30% dry matter.

FIG. 2 represents the flow curve for a steep solution obtained from deformulated gelatin capsules containing 30% dry matter.

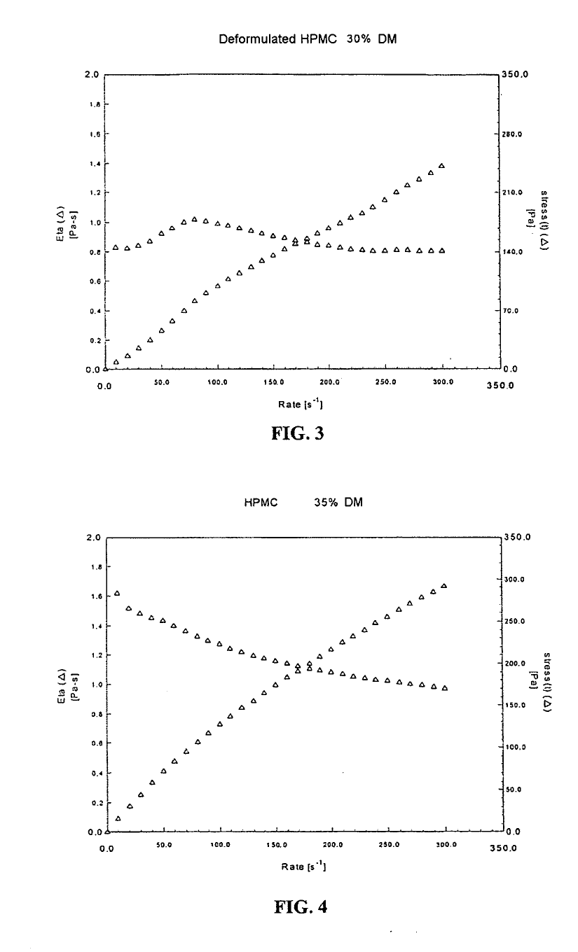

FIG. 3 represents the flow curve for a steep solution obtained from deformulated HPMC capsules containing 30% dry matter.

FIG. 4 represents the flow curve for an HPMC-based steep solution containing 35% dry matter.

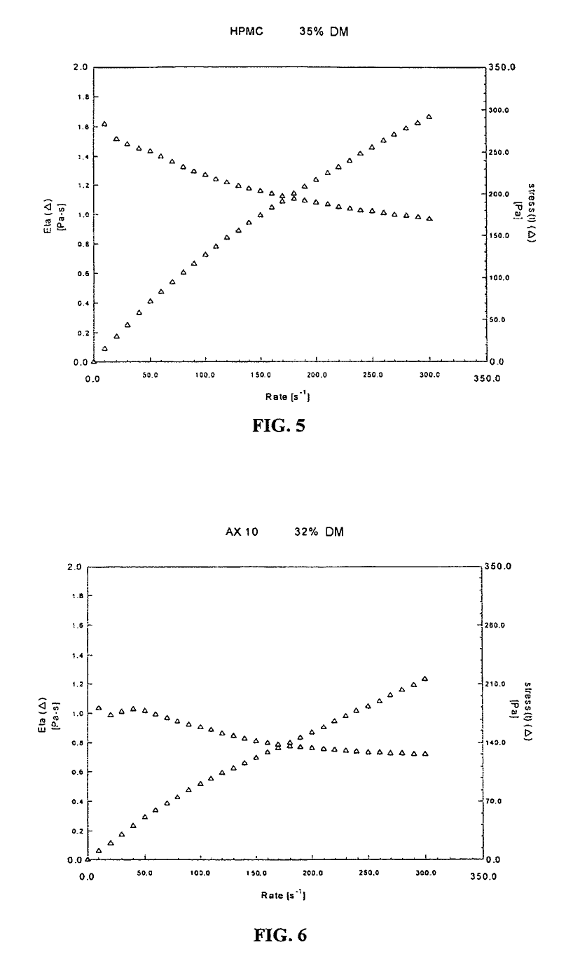

FIG. 5 represents the flow curve for an arabinoxylan-based steep solution containing 32.5% dry matter.

FIG. 6 represents the flow curve for an arabinoxylan-based steep solution containing 32% dry matter.

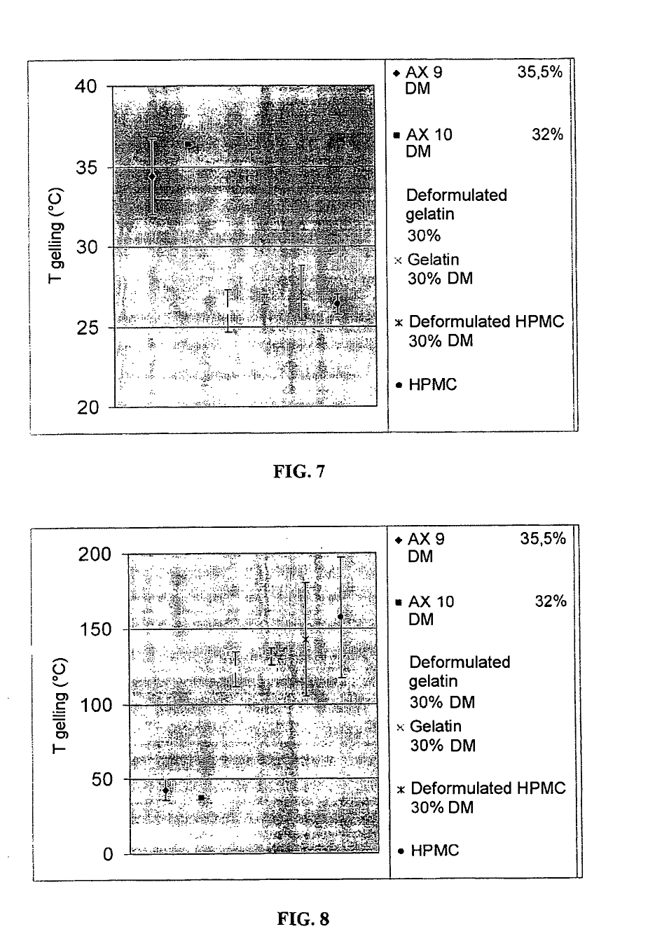

FIG. 7 represents the measurements of the gelling temperatures of various steep solutions.

FIG. 8 represents the measurements of gelling times of various steep solutions.

FIG. 9 represents the gelling profiles of an arabinoxylan-based steep solution containing 32.5% dry matter.

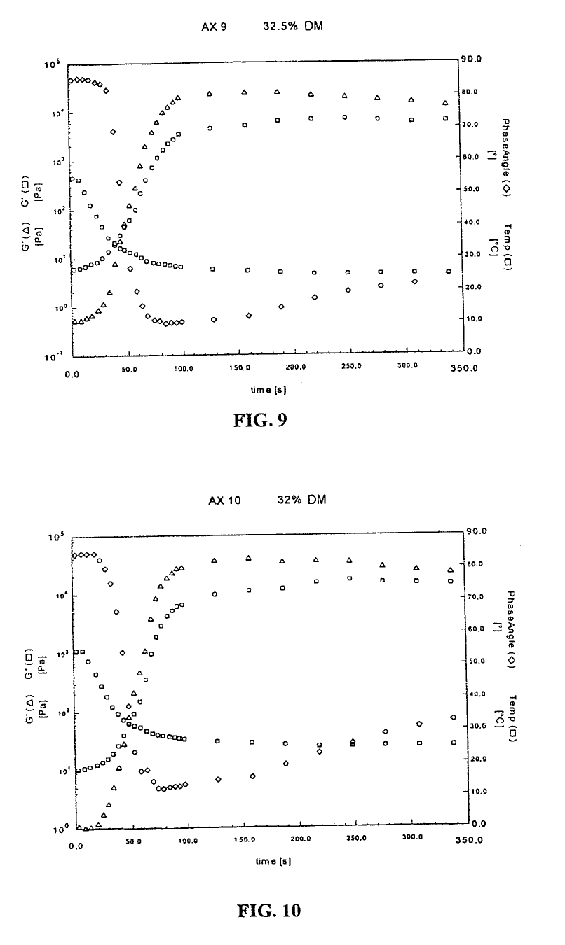

FIG. 10 represents the gelling profiles of an arabinoxylan-based steep solution containing 32% dry matter.

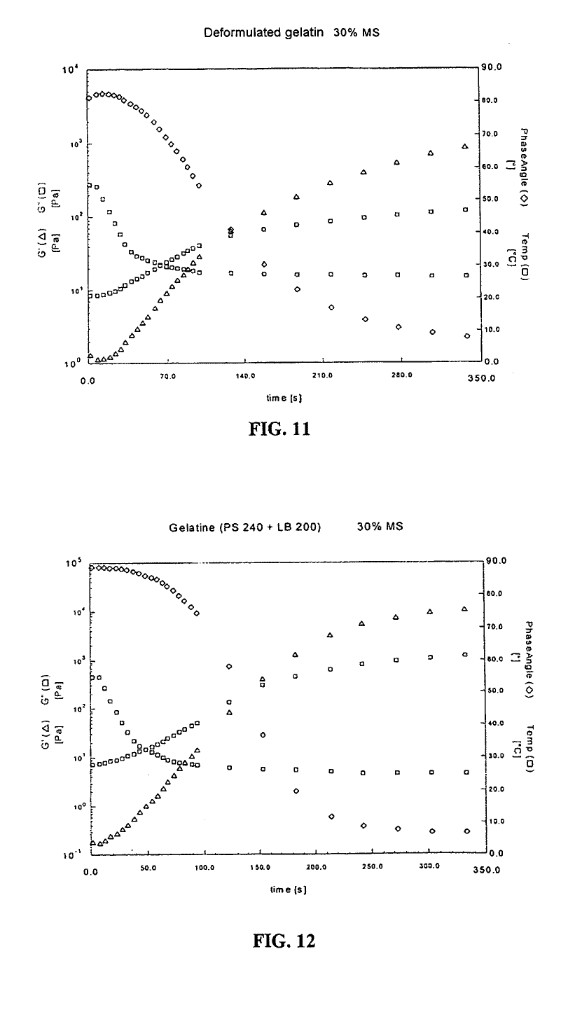

FIG. 11 represents the gelling profiles of a steep solution obtained from deformulated gelatin capsules containing 30% dry matter.

FIG. 12 represents the gelling profiles of a gelatin-based steep solution containing 30% dry matter.

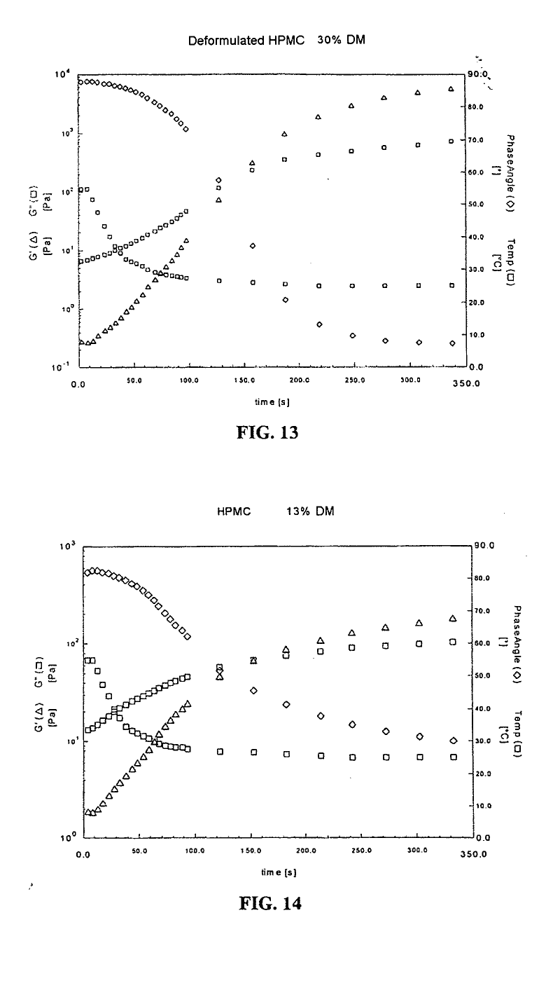

FIG. 13 represents the gelling profiles of a steep solution obtained from deformulated HPMC capsules containing 30% dry matter.

FIG. 14 represents the gelling profiles of an HPMC-based steep solution containing 13% dry matter.

### EXAMPLES:

### Example 1

### Composition by Weight of Heteroxylans Isolated from Corn Bran

The extraction of the heteroxylans is carried out according to the protocol described by Chanliaud et al. (Journal of cereal Science, 21, pp. 195-203, 1995). Variants have been introduced in order to obtain a method which can be exploited industrially and which allows access to the various grades of heteroxylans (grade “A”, “B” or “C” heteroxylans).

1) Preparation of the Grade “C” Heteroxylans

The corn bran heteroxylans are extracted in an alkaline medium (pH: 11-12), with lime (Ca(OH)₂ at saturation, 1.5 M potassium hydroxide) and at high temperature (about 90° C. to about 100° C. for two hours). A solid/liquid separation makes it possible to separate the heteroxylan-rich solution from a mixture consisting in particular of cellulose, proteins and carbohydrates. The solution is neutralized by adding acid, preferably sulfuric acid or hydrochloric acid.

A liquid extract of grade “C” heteroxylans is thus obtained, which may be concentrated in order to obtain a dry extract of about 15%. The extract thus obtained can then be dried, preferably spray-dried, so as to obtain a grade “C” heteroxylan powder containing from about 55% to about 70% by weight of grade “C” heteroxylans and a large quantity of salt (from about 10% to about 20%) and of other molecules such as polyphenols, tannins which can color the heteroxylans.

2) Preparation of the Grade “B” Heteroxylans

The liquid extract of grade “C” heteroxylans as obtained above, at the end of the steps of alkaline extraction, solid/liquid separation and neutralization, is subjected to a step of demineralization, by ultrafiltration, so as to obtain a liquid extract of grade “B” heteroxylans containing a salt level of less than 3%.

The liquid extract of grade “B” heteroxylans thus obtained or ultrafiltration retentate is then concentrated so as to obtain an extract of grade “B” heteroxylans containing about 15% heteroxylans by weight of dry matter. The extract thus obtained can then be dried, preferably spray-dried, so as to obtain a grade “B” heteroxylan powder containing about 71% to about 80% heteroxylans, said powder being slightly colored and still containing polyphenols.

3) Preparation of the Grade “A” Heteroxylans

The liquid extract of grade “B” heteroxylans containing a salt level of less than 3%, obtained as described above at the end of the steps of alkaline extraction, demineralization by ultrafiltration, is then purified by desalting and decolorization, with the aim of respectively removing the sediments present in the grade “B” heteroxylan extract and its light brown color predominantly linked to the presence of polyphenols.

Thus, after the demineralization step, the heteroxylans are purified by precipitation from ethanol or by successive passages over various ion-exchange resins and/or adsorption resins.

Other decolorization routes exist, in particular by using a powerful oxidizing agent of the hydrogen peroxide (H₂O₂) type. The method selected for the production of heteroxylans intended for the food-processing market does not use this type of agent, but uses routes which show regard for the consumer and the environment.

The heteroxylan extract obtained at the end of the purification step is dried, preferably spray-dried, so as to obtain a grade “A” heteroxylan powder having a grade “A” heteroxylan content greater than 81%. The white and neutral grade “A” heteroxylans correspond to highly purified products.

The compounds forming the purified heteroxylans are presented in the table below:

TABLE 1

| Compounds | % by weight |
|---|---|
| Arabinose | 26.4 |
| Xylose | 45.7 |
| Galactose | 7.5 |
| Glucuronic acid | 5.8 |
| Glucose | 2 |
| Starch | 1.1 |
| Proteins | 2.4 |
| Minerals | 3.7 |
| Others | 5.4 |

Among these grade “A” heteroxylans, it was the arabinoxylans (AX) which were used to produce the film-forming composition and the capsules according to the invention. These grade “A” AX have a molar mass of 250 000 g/mol.

It is also possible to envisage using grade “H” arabinoxylans. These arabinoxylans are obtained from grade “A” arabinoxylans by enzymatic hydrolysis using hemicellulases, in particular xylanases. These arabinoxylans have a molar mass of the order of 100 000 g/mol.

### Example 2

### Choice of the Control Capsules

The strategy for formulating the controls used is based both on:
the deformulation of commercial capsules, that is to say their dissolution in defined proportions, which makes it possible to obtain a steep solution close to that used industrially. They are the gelatin and HPMC capsules distributed by CAPSUGEL;
the formulation of capsules using commercial ingredients, approved by the pharmacopoeia, in agreement with the proportions indicated in the literature.

### Example 3

### Materials and Methods

3.1 Preparation of the steep solution

The expression steep solution is understood to mean the solution in which the supports for the manufacture of the capsules are immersed.

3.1.1 Equipment Used

The solution is prepared:
in a stainless steel beaker (diameter (Ø)=7.5 cm)+cover (thermal glass Petri dish Ø=8 cm)
with magnetic stirring (0-1300 rpm) using a magnetic bar
in a thermostatted water bath.

3.1.2 Mixing of the Ingredients

In the case of the deformulations, no problem is posed: the capsules are simply dissolved in a given quantity of water.

In the case of the formulations of capsules, particularly the most complex, the order of incorporation of the ingredients constituting the dry matter is important.

Indeed, different affinities of the ingredients for water are observed; now, the hydration of a strongly hydrophilic ingredient risks occurring at the expense of that of a less hydrophilic ingredient introduced into the solution before it: the less hydrophilic molecules tend to come closer, which induces poor homogeneity of the mixture, or even the formation of lumps.

Furthermore, a gelling agent cannot become hydrated to the maximum if other ingredients are already interacting with the water, which will have repercussions on the gelled network on cooling and therefore on the gel strength.
The ingredients should therefore preferably be introduced into the solution in the order of their affinity for water. In this case, it involves solubilizing:
   1) the gelling agent,
   2) the film-forming agent,
   3) the bulking agent.
The glycerol used as plasticizer is in liquid form. Soluble in water, it is therefore incorporated prior to the introduction of any powdered dry material into water (except in the case of gellan gum).

Solubilization of the Dry Material

To allow good hydration and solubilization of the ingredients introduced in powdered form, they must be allowed a so-called maturation period in line with the industrial process.

The ingredients must indeed undergo hydration before their solubilization, on the same principle as that mentioned above: if another ingredient is incorporated immediately after, the hydration phase is disrupted, which causes consequences on the solubilization.

These ingredients are therefore allowed to solubilize one by one for a lapse of time following their introduction into solution.

Compensation for the Water Evaporated

The cover covering the beaker is not sufficient to prevent all evaporation during the preparation of the steep solution. Now, the loss of water induces an increase in its viscosity and the increase in its gel strength upon cooling.

Given that it is vital to control these two parameters well, we chose to compensate for the water evaporated, which makes it possible to have good repeatability of the viscosities and gel strengths upon the manufacture of the capsules.

Thus, knowing the theoretical total mass of the steep solution prepared, it is therefore sufficient to measure the missing mass by weighing and to compensate for it by the adding of water.

3.2 Manufacture of Films by Spreading

3.2.1 Equipment Used

The films are produced with the aid of an automated spreader (Automatic Film Applicator 1137-SHEEN) allowing spreading at a controlled rate (40 mm/s), combined with a manual spreader for thin layer chromatography (DESAGA HEIDELBERG) allowing, for its part, the spreading thickness (0-2 mm) to be controlled.

The film-forming solution is poured into the reservoir and spread at the desired thickness. The supports for the films are glass plates (50×20 cm) coated beforehand with an adhesive polyvinyl chloride (PVC) film facilitating the detachment of the film.

3.2.2 Conditions for Implementation

The final thickness of the films should thus be 100-110 μm, which necessitates a regulation of the spreading thickness.

All the formulas not necessarily having the same proportions of dry matter (DM), nor the same water retention capacity at constant DM, the spreading thickness can therefore vary according to the products.

Two drops of a preservative, sodium sulfite, were added to the sample formulation in order to ensure good preservation thereof.

3.3 Manufacture of Hard Capsules by Draining

The conditions for industrial manufacture of the capsules were reproduced using supports serving as molds for the formation of capsules and by producing a system which allows vertical draining of the samples followed by their horizontal predrying.

The choice of the Supports

The supports selected are supports made of teflon and of stainless steel which are normally used industrially (production of male and female shells).

The Draining System

The support can move by rotating during the steps of immersion, draining and drying of the capsules:
The speed of rotation is set at 100 rpm (on the basis of the control samples of deformulated gelatin).
The rotation is brought about by a motor system integrally attached to a rod onto which the supports fit.
The immersion and the raising of the supports occur, on the other hand, manually, slowly and smoothly.

The transition from vertical to horizontal is allowed by manual rotation of the system on its axis: this step allows the product accumulated at the end of the capsule during draining to become uniformly distributed over the support.

The conditions for production of the capsules are summarized in Table 2 below.

TABLE 2

| Steps of the | | |
|---|---|---|
| protocol | Operating conditions | Equipment |
| Temperature for | 70° C. +/− 2° C. | Water bath No. 1 |
| preparing the | | |
| solution | | |
| Temperature of the | 53 +/− 2° C. | Water bath No. 2 |
| steep solution | | |
| Immersion | Immersion depth: | Teflon support. |
| | 3 cm +/− 0.5 cm. | Mechanical |
| | Reached in 3 s. | rotation at |
| | | 100 rpm. Manual |
| | | movements. |
| Steep time | 12 s | |
| Coming out of the | 3 s | |
| support | | |
| Rising - draining | Slow rising. | |
| | Keeping the support | |
| | 2-3 cm above the | |
| | solution. | |
| | Variable draining | |
| | time (Tₑ) | |
| | Tₑε] Tₑ₀, Tₑₘₐₓ [<2 min | |
| Horizontal | At Tₑ In 2 s. Rotation | |
| transition | 1 min. | |

The final thickness of the capsules depends directly on the draining time.

Now, the draining is determined by the viscosity of the solution combined with its kinetics of gelling: a solution with a high viscosity and a fast kinetics of gelling will thus become drained a lot less than a solution which is not very viscous and which gels slowly.

Between the time when the sample leaves the steep solution (Tₑ₀) and that when no drop falls through lack of product or because of gelling (Tₑₘₐₓ), it is possible to stop the draining by bringing the system to the horizontal.

3.4. Method of Drying

3.4.1 Equipment Used

Industrially, the drying is carried out:
at a few degrees above room temperature (22-28° C.),
at a relative humidity (RH) of between 35 and 85%.
   We therefore used a thermostatted ventilated oven with regulated RH (WTP Binder Labotechnik)

3.4.2 Drying Conditions

The drying conditions have a great effect on the appearance of the capsules (brittleness and thickness in particular) which depends directly on their water content. It is therefore vital to control the relative humidity during drying.

The drying is carried out at 30° C.-40% RH.

In the case of the films, it is often recommended to remove them from their PVC support before complete drying; indeed, that makes it possible both:
to avoid breaking the samples because of their possible fragility after drying;
to have less difficulty in detaching them when they have adhered strongly to the plate.

In the same manner, for the capsules, it is preferable to detach them as soon as possible from their teflon or stainless steel support. To allow easier detachment, the support may be oiled beforehand with dietary vegetable fats.

The minimum time for detaching the product, film or capsule, from its support is an important parameter to be evaluated because it gives a rough idea of its kinetics of drying.

Finally, at a controlled RH, the drying is complete when the water content of the capsules stabilizes.

The capsules, once dry, are packaged at room temperature in plastic bottles closed with a screw cap, which makes it possible to preserve their properties.

For the films, once dry, they are packaged in aluminum foil between two sheets of sulfurized paper so as not to stick.

The properties of these films and capsules are analyzed during and after manufacture, from the steep solution to the finished product.

### Example 4

### Formulation of the Control Capsules

4.1 Deformulation/Reformulation of the Commercial Capsules

The deformulation method consists in:
1) dissolving the gelatin or HPMC capsules with known characteristics in water,
2) trying to obtain capsules with the same characteristics by the established method of manufacture in a laboratory.

Indeed, the commercial gelatin and HPMC capsules may be mainly characterized by:
Their water content (X_{w} in %), between the interval [12-16%].
Their thickness (t in μm), between the interval [100-110 μm].

We therefore tried to obtain capsules which have characteristics which are not very different, if not similar.

The operating conditions which make it possible to obtain gelatin- and HPMC-based capsules similar to those available commercially are grouped together in Table 3 below:

TABLE 3

| Study parameters | Abbreviations | Gelatin | | HPMC | |
|---|---|---|---|---|---|
| Dry matter | % DM | 30% | | 30% | |
| Draining | tₑ | 40 | s | 40 | s |
| Detachment | t_{d} | ≧15 | min | ≧1 | h |
| Drying | tₛ | 2-3 | h | 3-4 | h |

| Thickness | t | 100-110 μm | |
|---|---|---|---|
| Water content | X_{w} | 11-12% | 11-12% |

4.2 Formulation from Commercial Ingredients

As above, the controls formulated with our ingredients should have the same properties as the commercial control capsules, in particular as regards appearance, water content and thickness. The formulations should therefore be in conformity therewith.

(a)-Formulation of the hard gelatin capsules
Choice of the gelatin

There are two types of gelatin, types A and B. Each of these gelatins may be independently used for the manufacture of capsules, but their combination is recommended in order to optimize their characteristics (AUGSBURGER, 1991).

Two gelatins with Bloom strength of between 150 and 280 Blooms were tested:
Gelatin PS 240, belonging to type A (Pig Skin 240 Blooms).
Gelatin LB 200, belonging to type B (Limed Bone 200 Blooms).

Choice of the Formulation

Numerous trials were carried out aimed at finishing the formulation which makes it possible to obtain capsules having characteristics which are closest to the commercial capsules.

The optimum formulation is presented in Table 4 below:

TABLE 4

| | Concentrations as | Concentrations as |
|---|---|---|
| Ingredients | dry matter (% w/w) | total mass (% w/w) |
| PS 240 | 71.25 | 21.4 |
| LB 200 | 23.75 | 7.1 |
| Glycerol | 5 | 1.5 |
| Volvic ® | — | 70 |

Glycerol is the plasticizer most widely used for the production of hard gelatin capsules and mineral water allows the repeatability of the results not to be influenced by a changing water quality.

The dry matter contents were adjusted according to the results obtained in the deformulation of the gelatin capsules.

(b) Formulation of the HPMC capsules

Choice of the HPMC

The HPMC used is the following: METHOCEL® ElS (DOW CHEMICAL®). This HPMC is authorized by the European pharmacopoeia.

Choice of the Formulation

In accordance with the trials carried out for the gelatin capsules, similar trials were carried out for the HPMC capsules so as to determine the formulation which made it possible to obtain capsules which are closest to those available commercially.

This formulation is presented in the following Table 5:

TABLE 5

| | Concentrations as | Concentrations as |
|---|---|---|
| Ingredients | dry matter (% w/w) | total mass (% w/w) |
| Methocel ® | 79.2 | 10.3 |
| Glycerol | 15.4 | 2 |
| Gelcarin ® | 5.4 | 0.7 |
| Ethanol | — | 22 |
| Volvic ® | — | 65 |

The additional gelling agent is the same as that selected for the arabinoxylan-based formulation.

### Example 5

### Formulation of Arabinoxylan-Based Capsules

Two different formulations of arabinoxylan-based capsules were produced and tested:
Formulation of hard AX capsules without maltodextrin.
Formulation of hard AX capsules with maltodextrin.

5.1 Formulation of hard AX capsules without maltodextrin 5.1.1: Concentrations tested Table 6 below presents the concentration ranges tested for the hard AX capsule formulation without maltodextrins.

TABLE 6

| | | | Concentrations |
|---|---|---|---|
| Ingredients | | Type | (% w/w) |
| Film-forming | AX | Quality A | 9-16 |
| agent | | | |
| Plasticizer | Glycerol | Anhydrous purity > | 0-25 |
| | | 98% | |
| Gelling | Carra- | GENULACTA ® Iota | 0.8-2 |
| agent | geenans | LP60 | |
| | | GELCARIN ® XP 3490 | 0.9-2.5 |
| | Pectins | GENU ® Pectin | 1-5 |
| | | Type B | |
| | | 150USA SAG Rapid | |
| | | set | |
| | | GENU ® Pectin LM | |
| | | 101 AS | |
| | Gellan | KELCOGEL ® | 0.08-0.2 |
| | | LT 100 HA | |
| Solvent | Water | Volvic ® | 60-80 |

5.1.2: Dissolution of AX

A quantity of less than 15% (total mass) of AX is easily dissolved in water (Volvic®) heated to 70° C. but it is in the region of 10% that the capsules are the most homogeneous.

As for gelatin, the high affinity of the AXs for water makes it possible:
to presolubilize them by rapid introduction into the solution,
and then to allow them to become completely solubilized, with stirring.

Unlike gelatin and HPMC, AXs do not pose any problem of overrun of the steep solution: the use of an antifoaming agent is not therefore a priori necessary industrially.

5.1.3: Results

Two Formulations were Selected:
the first, containing about 25% (w/w) glycerol makes it possible to produce soft AX capsules;
the second, containing 2.4% (w/w) glycerol gives, for its part, hard capsules with a homogeneous appearance and a soft texture.

These formulations are presented in Table 7 below:

TABLE 7

| | | Concentration | Concentration |
|---|---|---|---|
| | | as dry matter | as total mass |
| Formula | Ingredients | (% w/w DM) | (% w/w) |
| AX 37% DM 25% | AX “A” | 26.5 | 9.8 |
| glycerol | | | |
| | Glycerol | 66.4 | 24.6 |
| | Gellan | 0.3 | 0.1 |
| | Gelcarin ® | 6.8 | 2.5 |
| | Volvic ® | — | 63 |
| AX 18.4% DM | AX “A” | 75.6 | 13.9 |
| 2.4% glycerol | | | |
| | Glycerol | 13.3 | 2.4 |
| | Gelcarin ® | 11.1 | 2 |
| | Volvic ® | — | 81.6 |

Note:
The gellan gum is added to the AX formula 25% glycerol so as to slightly increase the viscosity of the hot solution.

We can therefore conclude therefrom that the AXs make it possible to manufacture not only hard capsules but also soft capsules by a simple formulation.

Like the main gelatin and HPMC controls, a draining time of 30 s makes it possible to manufacture hard capsules with a thickness of 100 μm.

The capsules can be detached from their support from 2 h 30 min-3 h, with a lot of ease.

As for the capsules formulated from HPMC, it can be considered that the drying is complete after 3-4 h.

The water content stabilizes around 9-10%, that is below that measured on the control samples (11% on average): this is due to a lower water retaining capacity of the “AX+Gelcarin®” combination than gelatin or HPMC.

5.1.3: Conclusion

The hard capsules more preferably studied have an appearance and a texture in every respect similar to the gelatin and HPMC controls. However, the DM content of the steep solutions is less than that of the controls, between 30 and 35% as total mass.

The AX- and Gelcarin®-based formulation being better defined, we sought to develop a formula incorporating a larger quantity of DM, by introducing bulking agents into the solution.

5.2 Formulation of Hard AX Capsules with Maltodextrin

5.2.1: Implementation

We chose to study two hard capsule formulations, one containing 10% glycerol, the other containing 12%, in order to compare the characteristics thereof. The protocol used for the preparation of the steep solution and the manufacture of the capsules is identical for the three formulas.

Table 8 below groups together the optimum AX-based formulations which make it possible to manufacture hard capsules with a high DM content (percentages rounded off).

TABLE 8

| | Concentrations | | | Concentrations | | |
|---|---|---|---|---|---|---|
| | as dry matter | | | as total | | |
| | (% w/w DM) | | | mass (% w/w) | | |
| | AX 3 | AX 9 | AX 10 | AX 3 | AX 9 | AX 10 |
| | 35% | 32.5% | 32% | 35% | 32.5% | 32% |
| Formulas | DM | DM | DM | DM | DM | DM |
| AX “A” | 25 | 25 | 25.6 | 8.8 | 8.1 | 8.2 |
| Glycerol | 12 | 12 | 10 | 4.25 | 3.9 | 3.2 |
| Gelcarin ® | 4 | 4 | 4.1 | 1.4 | 1.3 | 1.3 |
| DM DE 19 | 59 | 59 | 60.3 | 20.65 | 19.2 | 19.3 |
| Volvic | — | — | — | 65 | 67.5 | 68 |

5.2.2: Results

It is checked that the capsules prepared are similar in appearance and texture to the control capsules.

The results obtained for AX 9 show that a draining of 30 s makes it possible to obtain capsules which are in conformity. The results are similar for the AX 10 capsules, the 2 formulas differing little.

### Example 8

### Measurements of Viscosity and Kinetics of Gelling of the Steep Solutions

8.1 Measurements of Viscosity

The apparatus used is the Rheometric Scientific SR 5000. It is a rheometer which can carry out measurements both during flow and during stress oscillations (Dynamic Stress Rheometer).

Regardless of the type of measurement, however, the rotor used is a parallel plane type geometry with a diameter of 5 cm. On the other hand, depending on the measurement made, the air gap is different:
0.5 mm for the measurements during flow,
2 mm for the dynamic measurements.

8.1.1 Results

The mean viscosity results are given in the following Table 9:

TABLE 9

| | |
|---|---|
| | Viscosity (Pa · s) |
| Samples | at γ = 1s⁻¹ |

| | | |
|---|---|---|
| AX 2 | Mean | 3.29 |
| 32.5% DM | | |
| | CI | 2.48 |
| AX 3 | Mean | 4.41 |
| 32% DM | | |
| | CI | 14.90 |
| Deformulated gelatin | Mean | 4.15 |
| 30% DM | | |
| | CI | 3.5 |
| Gelatin 30% DM | Mean | 5.33 |
| | CI | 17.5 |
| Deformulated HPMC | Mean | 0.76 |
| 30% DM | | |
| | CI | 0.49 |
| HPMC | Mean | 1.47 |
| 13% DM | | |
| | CI | 2.01 |

Important note: the viscosities measured at 40° C. give a mean result of 900 mPa.s for AX 2. However, although this value is not exactly within the margins given by the literature at this temperature, we judged that they were too high (1000 and 8000 mPa.s) to be a valid reference. Accordingly, we chose to carry out the measurements at 55° C.: this value being that for the steep solution, the viscosities can be compared to the industrial production.

8.1.2 Discussion

Regardless of the product considered, the curves obtained are typical of a rheo-fluidizing behavior: the viscosity of the products decreases with the increase in stress.

However, various behaviors are manifested depending on the compositions, as represented in FIGS. 1 to 6.

The viscosity of the gelatin samples remains high at the low speed gradients (y) whereas the AX and HPMC solutions have a fairly low viscosity starting from the low stress values.

However, the AX and HPMC solutions have a viscosity which stabilizes rapidly unlike the gelatin solutions. Furthermore, it is observed that the gelatin solutions can enter into a turbulent regime starting from a speed gradient of 250 s⁻¹ (in general above 300 s⁻) whereas for AX and HPMC, the solutions withstand higher speed gradients (in general, entry into the turbulent regime does not occur before 600 s⁻¹).

The AX and HPMC solutions are therefore more stable to the high stresses than the gelatin solutions. The stress threshold above which the behavior of the solutions can be compared with a Newtonian behavior is less marked for the AX and HPMC solutions than for the gelatin solutions. This also manifests itself by a gradient value which tends toward 1.

The AX and HPMC solutions have a more constant reaction to stresses than gelatin.

The measurements performed on deformulated gelatin and AX 9 were repeated nine times whereas there were only three to six repeats for the other samples: this explains higher confidence intervals.

Moreover, this confidence interval is very important for HPMC whose formulation was produced (HPMC 35%). This can be explained by an increase of the viscosity of the solution during the measurements which is much higher than for the other samples. This phenomenon can be explained by an evaporation of the ethanol.

Overall, a viscosity of the steep solutions at 55° C. is therefore measured which is similar for our different products: it ranges between 500 and 900 mPa.s.

The AX samples have, however, a viscosity which is substantially lower than that of the gelatin solutions: if our most repeatable measurements are considered (AX 9 compared with deformulated gelatin), these viscosities are 600-800 mPa.s for AX against 700-900 mPa.s for gelatin.

In parallel, we also studied the kinetics of gelling of our solutions.

8.2 Kinetics of gelling

By definition, two parameters are evaluated by measuring the kinetics of gelling:
the mean gelling time of the solutions,
the temperature at which this gelling occurs.

8.2.1 Gelling Time and Temperature

These measurements were carried out on Rheometrics on all the samples. FIGS. 1 and 2 represent respectively the mean gelling times and the mean gelling temperatures.

FIGS. 3 to 8 represent the gelling profile as a function of time for each of the compositions tested.

2.3 Discussion

The cooling conditions being similar to the production of capsules and under Rheometrics measurements, analogy between the measurement of the time of onset of gelling and that of the maximum draining time is verified: indeed, during the production of the capsules, it is possible to allow the HPMC and gelatin solutions to drain for slightly less than 2 minutes whereas the AX solutions no longer drain after 50 s, which is measured here.

The kinetics of cooling being the same for all the samples, a higher gelling temperature is therefore obtained in parallel for the AX solutions and for the controls.

A higher maximum gel strength (G′) can be measured for our AX solutions than for the controls (20 to 40 000 Pa against 10-20 000 for the deformulated controls in particular). However, our AX gels, which formed more rapidly than the gelatin and HPMC gels, take longer to stabilize at room temperature, which explains a further reduction in G′: the gel strength stabilizes after 5 minutes at 25° C. around the same values as for the controls, that is 10 to 20 000 Pa.

8.3 Conclusion

We have thus been able to manufacture AX-based capsules which have the same appearance, texture and thickness properties as the commercial controls.

The method of production is then substantially similar: only the draining period necessary for the manufacture of capsules of standard thickness is slightly lower for the AX solutions, the other manufacturing parameters remaining unchanged.

This difference is explained by a lower viscosity of the AX solutions compared with the control solutions. Moreover, this low viscosity is compensated for by a much higher kinetics of gelling, which can be substantially improved by adjusting the gelling power of the gelling agent used.

The formulation of our products being optimized for a standard capsule thickness, we then sought to complete this optimization on the criterion of water content.

### Example 9

### Comparison of the Mechanical and Dissolution Properties of the Hard Capsules

We logically chose to continue to characterize the capsules obtained by the formulas AX 9 and AX 10, for which the study was in the greatest detail.

The mechanical and dissolution properties of these capsules were measured and, in some cases, compared with those of the control gelatin and HPMC capsules by studying both capsules and films.

9.1 Mechanical Properties

The study was carried out on films produced by spreading using steep solutions at 70° C.

In the same manner as was established for each formula which draining time makes it possible to obtain a standard capsule thickness, we sought which spreading thickness makes it possible to manufacture films of standard thickness.

9.1.1 Spreading thicknesses allowing standardization of the thickness of the films after drying

We sought to use spreading thicknesses which make it possible to obtain, after drying, thicknesses equivalent to the standards for capsules, that is thicknesses of between 100 and 110 μm.

The results obtained are presented in the following Table 10:

TABLE 10

| | | | De- | | De- | |
|---|---|---|---|---|---|---|
| | AX 9 | AX 10 | formulated | Gelatin | formulated | HPMC |
| Samples/ | 32.5% | 32% | gelatin | 30% | HPMC | 13% |
| Thicknesses | DM | DM | 30% DM | DM | 30% | DM |
| Spreading | 600-700 | 650-750 | 600 | 600-700 | 600-700 | 1250-1500 |
| thickness | | | | | | |
| (μm) | | | | | | |
| Thickness | 100-120 | 100-130 | 105-110 | 100-115 | 100-115 | 100-130 |
| after | | | | | | |
| drying | | | | | | |
| (μm) | | | | | | |

On average, the films obtained have a thickness of 110-115 μm. Only the HPMC-based films containing 13% DM have a thickness after drying of between 100 and 130 μm.

9.1.2 Mechanical properties of the films

From the films obtained, we therefore carried out measurements of mechanical properties on Instron 1122.

The results are presented in the following Table 11:

TABLE 11

| | Rupture | | Elasticity | | Deformation | |
|---|---|---|---|---|---|---|
| Parameters/ | strength (N) | | (N · s⁻¹) | | (%) | |
| Samples | Mean | C.I. | Mean | C.I. | Mean | C.I. |
| AX 9 32.5% DM | 31 | 3 | 18 | 2 | 5.2 | 1.3 |
| AX 10 32% DM | 62 | 11 | 56 | 9 | 4 | 0.4 |
| HPMC 13% DM | — | — | 50 | 7 | 17.0 | 4.2 |
| Gelatin 30% DM | — | — | 125 | 6 | 1.7 | 0.8 |
| Formulated | — | — | 103 | 23 | 1.2 | 0.2 |
| gelatin | | | | | | |
| 30% DM | | | | | | |
| Deformulated | — | — | 118 | 6 | 1.5 | 0.4 |
| HPMC | | | | | | |
| 30% DM | | | | | | |

It is observed that the elasticity of the AX 10-based film is better than that of the commercial HPMC-based film. On the other hand, it is not as good as that of the gelatin-based films.

The deformation of the AX 10-based film is on the other hand better than that of the deformulated HPMC- and gelatin-based films. It is on the other hand lower than the deformation of the commercial HPMC-based film. However, it is important to note that the latter has a thickness of 130 μm, which can explain why it possesses such a high deformation capacity.

9.1.3 Conclusion

It would be advantageous to carry out measurements at the correct film thickness on the HPMC 13% DM sample, formulated according to the same mode as our AX samples (without maltodextrin). Indeed, the results obtained for elasticity at 130 μm are slightly greater than those obtained for AX 10 at 115 μm. It is therefore possible that these new measurements give results similar to those obtained for AX 10.

9.2 Kinetics of disintegration and dissolution The first measurements carried out show that the capsules produced based on AX satisfy the dissolutest and have moreover better dissolution properties than the gelatin capsules.

Conclusion:

Arabinoxylans therefore constitute substitutes of choice for the industrial production of pharmaceutical hard capsules.

Indeed, we were able to define a formulation from natural ingredients of plant origin, based on the combination of arabinoxylans and a gelling agent, Gelcarin®: this formulation makes it possible to obtain hard capsules for a dry matter content and according to a method which are identical to those used for the commercial samples.

These capsules have characteristics similar to the industrial controls.

The capsules obtained have an appearance, a texture and sizes after drying which are similar to the standard industrial values.

For the desired texture, their water content stabilizes at around 9%, that is a value less than the usual industrial values but at which the gelatin and HPMC capsules are usually brittle. This property is advantageous for the behavior during storage.

Finally, it is notable that our capsules have better dissolution properties than gelatin. In this context, their mechanical properties may be improved by increasing their thickness.

The ease with which soft capsules can be produced from arabinoxylans gives an indication of the excellent quality of the hard capsules which may be obtained by working in greater detail on the method and by developing the formulation produced.

1-20. (canceled).

21. A film-forming composition for the manufacture of capsules, comprising:
at least one compound of the heteroxylan type,
at least one plasticizing agent, and
at least one gelling agent.

22. The composition of claim 21, wherein the heteroxylan is arabinoxylan.

23. The composition of claim 21, wherein the plasticizer is chosen from the group of (poly)hydroxylated compounds, and more preferably still from the group comprising glycerol, sorbitol, polyethylene glycol, propylene glycol, maltitol, triacetin or mixtures thereof.

24. The composition of claim 21, wherein the gelling agent is selected from the group comprising carrageenans (κ- and ι-carrageenans), gellan gum, pectins or mixtures thereof.

25. The composition of claim 24, containing:
from 20 to 99% by dry weight of heteroxylan,
from 5 to 40% by dry weight of plasticizer, and
from 0.1 to 20% by dry weight of gelling agent.

26. The composition of claim 21, further comprising at least one bulking agent.

27. The composition of claim 26, wherein the bulking agent is chosen from the group comprising carbohydrates such as sucrose, fructose, starch, cellulose, maltodextrins, cereal and noncereal flours, mineral fillers such as calcium, sodium or potassium salts or mixtures thereof, and preferably a maltodextrin having a Dextrose Equivalent value of 5 to 40.

28. The composition of claim 27, containing from 0 to 70% by dry weight of bulking agent.

29. The composition of claim 21, wherein said compound(s) of the heteroxylan type is(are) preferably extracted from corn, rye or rice brans, or mixtures thereof.

30. The composition of claim 21, wherein said compound(s) of the heteroxylan type has(have) a molar mass of between 100 000 and 300 000 g/mol.

31. The composition of claim 21, further comprising an additive or a mixture of additives chosen from:
colorings, chosen in particular from the group consisting of titanium oxide, iron oxide, patent blue, quinoline yellow, orange yellow S, cochineal red A or chlorophillin-copper complex,
antioxidants such as ascorbic acid, tocopherol, butylhydroxyanisole (BHA) or butyhydroxytoluene (BHT).

32. The composition of claim 31, comprising:
between 0 and 3% by dry weight of coloring, and/or
between 0 and 3% by dry weight of antioxidant.

33. The composition of claim 21, wherein it is in the form of a solution, preferably an aqueous solution.

34. The composition of claim 33, containing from 25 to 80% by weight of water.

35. A method for producing film from the composition of claim 21.

36. A film prepared from the composition of claim 21.

37. The film of claim 36, having the following mechanical properties:
a rupture strength of between 30 and 250 N,
an elasticity of between 20 and 120 N.s-1,
a deformation of between 2 and 20%.

38. A capsule prepared from a composition of claim 21.

39. A soft capsule, comprising:
between 60 and 99% by dry weight of at least one heteroxylan,
between 5 and 40% by dry weight of at least one plasticizer,
between 0.1 and 20% by dry weight of at least one gelling agent,
between 0 and 70% of at least one bulking agent.

40. A hard capsule, comprising:
between 20 and 90% by dry weight of at least one heteroxylan,
between 10 and 30% by dry weight of at least one plasticizer,
between 5 and 20% by dry weight of at least one gelling agent,
between 0 to 70% by dry weight of at least one bulking agent.
